(19) United States
(12) Patent Application Publication
(10) Pub. No.: US 2023/0301969 A1
(43) Pub. Date: Sep. 28, 2023

(54) DOSING SCHEDULE FOR A METHOD OF TREATMENT WITH DHODH INHIBITORS

(71) Applicant: Bayer Aktiengesellschaft, Leveerkusen (DE)

(72) Inventor: Andreas JANZER, Berlin (DE)

(73) Assignee: Bayer Aktiengesellschaft, Leveerkusen (DE)

(21) Appl. No.: 18/023,318

(22) PCT Filed: Aug. 24, 2021
(86) PCT No.: PCT/EP2021/073320

§ 371 Date: Feb. 24, 2023

(30) Foreign Application Priority Data
Aug. 25, 2020 (EP) 20192551.8

(51) Int. Cl. A61K 31/4196; A61P 35/00

(52) U.S. Cl. CPC A61K 31/4196; A61P 35/00

(57) ABSTRACT

The present invention provides N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide for use in a method of cancer treatment using a dose schedule of daily dosing on equal to 4 days up to 5 days followed by a short off period of equal to 2 days up to 3 days, as well as a method of treatment of cancer diseases with a composition comprising said DHODH inhibitor using the new dosing schedule.

The present invention provides a dosing schedule useful in a method of treatment of hyperproliferative and/or inflammatory disorders, more specifically cancer, with DHODH inhibitors, especially with N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide as well as a method of treatment of hyperproliferative and/or inflammatory disorders, more specifically cancer, with a composition comprising said inhibitors using the new dosing schedule.

### BACKGROUND

Acute myeloid leukemia (AML) is the most common acute leukemia in humans with a 5 year survival of only about 30%. AML is a malignancy of the myeloid line of blood cells. The incidence rates and chances of cure are highly age dependent. The chemotherapy standard of care for AML has not changed significantly over the last decades highlighting the need for novel therapies. A major hallmark of AML is differentiation arrest of the leukemic cells at early stages of cellular differentiation. The potential of leukemic differentiation therapy can be seen with the success of ATRA or arsenic trioxide inducing differentiation in acute promyelocytic leukemia (APL). Around 10% of AML belong to the APL subtype where leukemic cells are harbouring a chromosomal translocation resulting in fusions of oncoproteins involving the retinoic acid receptor. While treatment with ATRA or arsenic trioxide leads to a dramatic increase of patient survival, with overall survival rates of over 70%, unfortunately a comparable differentiation therapy for the non-APL AMLs is lacking (Management of acute promyelocytic leukemia: recommendations from an expert panel on behalf of the European Leukemia Net, Sanz M. A. et al, Blood 2009, 113(9), 1875-1891). Therefore new therapies inducing differentiation of AML cells are of high interest and medical need.

Dihydroorotate Dehydrogenase (DHODH) is located in the mitochondria of a cell and is the enzyme responsible for the fourth and rate limiting step in de novo pyrimidine synthesis converting dihydroorotate to orotate (Dihydroorotate-ubiquinone oxidoreductase links mitochondria in the biosynthesis of pyrimidine nucleotides, Löffler, M. et al, Molecular and Cellular Biochemistry 1997, 174, 125-129).

The pyrimidine synthesis is essential for most cancer cells to proliferate and it was recently also linked to differentiation of myeloid malignancies.

Inhibition of DHODH therefore has emerged as a potential treatment for several malignancies such as e.g. breast cancer, SCLC, liver cancer. Currently, inhibition of DHODH with small molecule inhibitors is under clinical investigation for hematological malignancies and lymphomas (NCT03451084, NCT03761069, NCT03760666; NCT03404726; NCT03834584). In the past DHODH inhibitors have also been tested in solid cancer indications using weekly doses or e.g. 5 days on 23 days off schedules for the DHODH inhibitor Brequinar sodium, however they were mainly found to be ineffective. Whereas in WO 2017/037022 a dosing of less than or equal to every 72 hours or less than or equal to every 48 h for three DHODH inhibitors including Brequina is disclosed, in WO20123241 a multiphasic dosing regimen is offered for Brequinar for the treatment of cancer but not supported by data and no further clinical trial information is available potentially supporting success of said dosing regimen. This underlines the difficulty to find a dosing schedule for these effectively DHODH inhibiting compounds in order to successfully treat patients suffering from cancer diseases. N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide (BAY 2402234) is a novel highly potent and selective DHODH inhibitor disclosed in (S. Christian et al, Leukemia. 2019; 33 (10):2403-15, and WO2018/077923). BAY 2402234 has previously been used in daily dosing schedules in preclinical models and initial clinical studies.

### SUMMARY

It has now been found, and this constitutes the basis of the present invention, that the schedules for the administration of the DHODH inhibitor compound N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide (BAY 2402234) show that short off phases of the treatment ensure equal efficacy in preclinical mouse xenograft models, while yielding higher exposure on treatment days. In short, a 4 days on 3 days off schedule as well as a 5 days on 2 days off schedule of BAY 2402234 administration has surprisingly been found to establish comparable efficacy to daily BAY 2402234 dosing, while achieving similar or even higher weekly doses (comparing the total sum of doses given over a 7 day period independent of schedule) however also allowing off treatment days for potential side effect recovery.

### Definitions

The term “DHODH inhibitor” includes small molecules inhibiting dihydroorotate dehydrogenase, such as e.g. PTC299, Aslan-003 and IMU-838, 6-fluoro-2-(2′-fluoro-[1,1′-biphenyl]-4-yl)-3-methylquinoline-4-carboxylic acid) (brequinar); 5-methyl-N-[4-(trifluoromethyl) phenyl]-isoxazole-4-carboxamide) (leflunomide); (2Z)-2-cyano-3-hydroxy-N-[4-(trifluoromethyl)phenyl]but-2-enamide)(teriflunomide), which can be made according to known methods. The DHODH inhibitors disclosed in WO2018/077923, especially in claims 8 and 9 therein, particularly BAY 2402234 which is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide a tautomer, an N-oxide, a salt, a salt of a tautomer or a salt of an N-oxide thereof, can be obtained by methods as disclosed in said WO2018/077923.

The term “daily treatments of equal to 4 days (96 hrs) up to 5 days (120 hrs) followed by a short off period of equal to 2 days (48 hrs) up to 3 days (72 hrs)” includes a treatment for four days or five days and a following off treatment period of two days or three days, as well as treatment for 96 hrs to 120 hrs followed by a short off period of equal to 48 hrs to 72 hrs and all combinations thereof. Whenever days are mentioned the shortest unit to count is one day whereas when hours are mentioned the shortest unit is one hour. The schedule can also be expressed as e.g. “four days or five days on/two or three days off” and all combinations thereof. The dose for an “on” day may be administered as most suitable for the individual getting the dose, e.g. once daily or splitted into one ore more doses per day.

If specific steps are mentioned, the term “comprising” used in the claims includes also the respective method having only the specific steps mentioned.

The term “hyperproliferative disease” includes, but is not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include sarcomas, and haematological malignancies including but not limited to leukemias, lymphomas, multiple myelomas.

Examples of “breast cancer”s include, but are not limited to, invasive ductal carcinoma, invasive lobular carcinoma, ductal carcinoma in situ, and lobular carcinoma in situ.

Examples of “cancers of the respiratory tract” include, but are not limited to, small-cell and non-small-cell lung carcinoma, as well as bronchial adenoma and pleuropulmonary blastoma and carcinoid tumor.

The term “lung cancer” includes all kinds of carcinoma of the lung, including, but not limited to, small-cell and non-small-cell lung carcinoma, pleuropulmonary blastoma and carcinoid tumor, more especially small cell lung cancer and non-small cell lung cancer.

Examples of “brain cancers” include, but are not limited to, brain stem and hypothalmic glioma, glioblastoma, gliosarcoma, glioma, cerebellar and cerebral astrocytoma, medulloblastoma, ependymoma, as well as neuroectodermal and pineal tumour.

The term “reproductive organs” includes male and female reproductive organs. Tumours of the male reproductive organs include, but are not limited to, prostate and testicular cancer. Tumours of the female reproductive organs include, but are not limited to, endometrial, cervical, ovarian, vaginal, and vulvar cancer, as well as sarcoma of the uterus.

“Tumours of the digestive tract” include, but are not limited to, anal, colon, colorectal, oesophageal, gallbladder, gastric, pancreatic, rectal, small-intestine, and salivary gland cancers.

“Tumours of the urinary tract” include, but are not limited to, bladder, penile, kidney, renal pelvis, ureter, urethral and human papillary renal cancers.

Eye cancers include, but are not limited to, intraocular melanoma and retinoblastoma.

Examples of “liver cancers” include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

“Skin cancers” include, but are not limited to, squamous cell carcinoma, Kaposi's sarcoma, malignant melanoma, Merkel cell skin cancer, and non-melanoma skin cancer.

“Head-and-neck cancers” include, but are not limited to, laryngeal, hypopharyngeal, nasopharyngeal, oropharyngeal cancer, lip and oral cavity cancer and squamous cell.

“Hematological cancer” includes but is not limited to malignancies originating from blood cells, especially white blood cells, more particularly leukemia, lymphoma and myeloma.

“Leukemias” include, but are not limited to acute lymphoblastic leukemia, acute myeloid leukemia, (acute) T-cell leukemia, acute lymphoblastic leukemia, acute lymphocytic leukemia (ALL), acute monocytic leukemia (AML), acute promyelocytic leukemia (APL), bisphenotypic B myelomonocytic leukemia, chronic lymphocytic leukemia, chronic myelogenous leukemia, chronic myeloid leukemia (CML), chronic myelomonocytic leukemia (CMML), large granular lymphocytic leukemia, plasma cell leukemia, and also myelodysplastic syndrome (MDS), which can develop into an acute myeloid leukemia.

“Lymphomas” include, but are not limited to, AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell lymphoma DLBCL, double-hit lymphoma and double-expressor lymphoma; anaplastic large cell lymphoma, B-cell lymphoma, cutaneous T-cell lymphoma, Burkitt's lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma and Sezary syndrome.

Cancer may be expressed as a solid tumor that may exist in any part or organ of the human body including but not limited to breast, respiratory tract, brain, reproductive organs, digestive tract, urinary tract, eye, liver, skin, head and neck, thyroid, parathyroid and their distant metastases.

The term “pharmaceutically acceptable excipient” means inter alia,
fillers and carriers (for example cellulose, microcrystalline cellulose (such as, for example, Avicel®)), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxyethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®)); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®))),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

### BRIEF DESCRIPTION OF THE DRAWINGS

FIG. 1:

FIG. 1 shows the development of tumor volume over time for the acute myeloid leukemia xenograft model THP1. 16 days post cell inoculation and after randomisation based on tumor size, administration of vehicle compared to daily dosing of 5 mg/kg p.o. and 1 Omg/kg in 4 days on, 3 days off p.o. schedule of BAY 2402234 started. After 7 days of treatment the vehicle control group showed a tripled tumor volume while both BAY 2402234 treatment arms led to a comparable stable disease with basically no further tumor growth after treatment initiation. The weekly dose for the daily schedule sums up to 35 mg/kg, while the weekly dose for the 4 days on, 3 days off schedule sums up to 40 mg/kg.

FIG. 2 shows the development of tumor volume over time for the Mantle cell lymphoma xenograft model Z138. Administration started at day 20 after tumor randomisation comparing vehicle treatments to (1) daily dosing of 5 mg/kg p.o. (2) 1 Omg/kg in 4 days on, 3 days off p.o., (3) 5 mg/kg 5 days on, 2 days off p.o. schedule of BAY 2402234. All three BAY 2402234 schedules showed comparable tumor regression in this model while using weekly doses of (1) 35 mg/kg, (2) 40 mg/kg and (3) 25 mg/kg.

FIG. 3 shows the development of tumor volume overtime for the colorectal cancer xenograft model HT29. Administration started at day 9 after tumor randomisation comparing vehicle treatments to (1) daily dosing of 5 mg/kg p.o. (2) 1 Omg/kg in 5 days on, 2 days off p.o. (3) 5 mg/kg 5 days on, 2 days off p.o. schedule of BAY 2402234. All three BAY 2402234 treatments led to tumor growth inhibition, which was comparable for the two 5 mg/kg treatments and slightly stronger in the 10 mg/kg treatment.

Throughout the experiment the bodyweight was measured and bodyweight behaved comparable throughout the different treatment schedules as shown in FIG. 4.

FIG. 4 shows the body weight of vehicle and BAY 2402234 treated animals in the HT29 xenograft model. No significant changes in the body weight. While the vehicle treated animals as well as the 5 mg/kg 5 days on 2 days off schedule had a relative constant bodyweight throughout the course of the experiment, the 10 mg/kg 5 days on 2 days off showed around 4% body weight loss at the end of the experiment and the 5 mg/kg daily schedule had a body weight loss at experiments end of above 10% indicating that off-treatment days have the potential to reduce side effects.

### DETAILED DESCRIPTION

The results shown in the experimental section indicate that an intermittent dosing schedule with 2 or 3 off days/week of a DHODH inhibitor can be equally potent to a daily dosing of the same inhibitor with similar or even higher weekly doses. At the same time the intermittent schedule is at least equally tolerated using a similar or even higher weekly dose. Given the higher daily doses in the intermittent schedule and thereby expected higher exposure peaks on treatment days this is a surprising finding. Thereby this should allow to avoid potential adverse events in a clinical setting by allowing patients rest days and a different exposure profile e.g. higher exposure peaks on the “on treatment days” due to a higher dose on a given day compared to the equally stretched daily dosing as demonstrated in mice example 3 (FIG. 4).

This was somehow unexpected as interruption of dosing is believed to be one major reason the solid cancer trials with DHODH inhibitor Brequinar (which was given weekly or in a 5 days on 23 days off schedule) failed to show sufficient clinical activity.

Thus a first aspect the invention provides a method of treatment of a hyperproliferative disease comprising administering a DHODH inhibitor with a schedule using daily treatments of equal to 96 hrs up to 120 hrs followed by a short off period of equal to 48 hrs up to 72 hrs and a subsequent repetition of the schedule as often as deemed necessary.

In an embodiment the invention provides a method of treatment of a hyperproliferative disease comprising administering a DHODH inhibitor with a schedule using daily treatments of equal to 96 hrs followed by a short off period of equal to 48 hrs and a subsequent repetition of the schedule as often as deemed necessary.

In an embodiment the invention provides a method of treatment of a hyperproliferative disease comprising administering a DHODH inhibitor with a schedule using daily treatments of equal to 96 hrs followed by a short off period of equal to 72 hrs and a subsequent repetition of the schedule as often as deemed necessary.

In an embodiment the invention provides a method of treatment of a hyperproliferative disease comprising administering a DHODH inhibitor with a schedule using daily treatments of equal to 120 hrs followed by a short off period of equal to 48 hrs and a subsequent repetition of the schedule as often as deemed necessary.

In an embodiment the invention provides a method of treatment of a hyperproliferative disease comprising administering a DHODH inhibitor with a schedule using daily treatments of equal to 120 hrs followed by a short off period of equal to 72 hrs and a subsequent repetition of the schedule as often as deemed necessary.

In an embodiment the invention provides a method of treatment of a hyperproliferative disease comprising administering a DHODH inhibitor with a schedule using daily treatments for 4 days followed by a short off period of 2 days and a subsequent repetition of the schedule as often as deemed necessary.

In an embodiment the invention provides a method of treatment of a hyperproliferative disease comprising administering a DHODH inhibitor with a schedule using daily treatments for 4 days followed by a short off period of 3 days and a subsequent repetition of the schedule as often as deemed necessary.

In an embodiment the invention provides a method of treatment of a hyperproliferative disease comprising administering a DHODH inhibitor with a schedule using daily treatments for 5 days followed by a short off period of 2 days and a subsequent repetition of the schedule as often as deemed necessary.

In an embodiment the invention provides a method of treatment of a hyperproliferative disease comprising administering a DHODH inhibitor with a schedule using daily treatments for 5 days followed by a short off period of 3 days and a subsequent repetition of the schedule as often as deemed necessary.

In further embodiments the DHODH inhibitor is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide a tautomer, an N-oxide, a salt, a salt of a tautomer or a salt of an N-oxide thereof which is used in the schedules mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments of equal to 96 hrs up to 120 hrs followed by a short off period of equal to 48 hrs up to 72 hrs and a subsequent repetition of the schedule as often as deemed necessary in order to control the disease, achieve its regression or cure the disease mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments of equal to 96 hrs followed by a short off period of equal to 48 hrs and a subsequent repetition of the schedule as often as deemed necessary in order to control the disease, achieve its regression or cure the disease mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments of equal to 96 hrs followed by a short off period of equal to 72 hrs and a subsequent repetition of the schedule as often as deemed necessary in order to control the disease, achieve its regression or cure the disease mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments of equal to 120 hrs followed by a short off period of equal to 48 hrs and a subsequent repetition of the schedule as often as deemed necessary in order to control the disease, achieve its regression or cure the disease mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments of equal to 120 hrs followed by a short off period of equal to 72 hrs and a subsequent repetition of the schedule as often as deemed necessary in order to control the disease, achieve its regression or cure the disease mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments for 4 days followed by a short off period of 2 days and a subsequent repetition of the schedule as often as deemed necessary in order to control the disease, achieve its regression or cure the disease mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments for 4 days followed by a short off period of 3 days and a subsequent repetition of the schedule as often as deemed necessary in order to control the disease, achieve its regression or cure the disease mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments for 5 days followed by a short off period of 2 days and a subsequent repetition of the schedule as often as deemed necessary in order to control the disease, achieve its regression or cure the disease mentioned herein.

In some embodiments the invention provides a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide with a schedule using daily treatments for 5 days followed by a short off period of 3 days and a subsequent repetition of the schedule as often as deemed necessary in order to control the disease, achieve its regression or cure the disease mentioned herein.

In some embodiments the invention provides a method of treatment of a hyperproliferative disease comprising administering a DHODH inhibitor with a schedule using daily treatments for 5 days followed by a short off period of 2 days and a subsequent repetition of the schedule as often as deemed necessary in order to control the disease, achieve its regression or cure the disease mentioned herein.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 4 to 5 days and
step b) pausing treatment for 2 to 3 days and
step c) optionally repeating the schedule defined by steps a) followed by step b) one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 4 to 5 days and
step b) pausing treatment for 2 to 3 days and
step c) optionally repeating the schedule defined by steps a) followed by step b) one or more times
whereby during and/or after treatment the effect of treatment is readily controlled.

In some embodiments the methods of such controlling the effect of treatment are selected from measuring the level of DHO in a blood sample, measuring the count ratio of normal cells to differentiation arrested cells in a blood sample, and non-invasive methods of imaging a solid tumor enabling measuring the seize of the remaining tumor and comparing to earlier measurement. Differentiation arrest can be measured as published in S. Christian et al, Leukemia, 33, 2403-2415 (2019) and assay 13 disclosed in WO2018/077923.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 4 to 5 days and
step b) pausing treatment for 2 to 3 days and
step c) optionally repeating the schedule defined by steps a) followed by step b) one or more times
whereby during and/or after treatment the effect of treatment is readily controlled by measurement of DHO in a blood sample and/or by measuring the count ratio of normal cells to differentiation arrested cells in a blood sample and/or by non-invasive methods of imaging a solid tumor enabling measuring the seize of the remaining tumor.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 4 to 5 days and
step b) pausing treatment for 2 to 3 days and
step c) optionally repeating the schedule defined by steps a) followed by step b) one or more times
whereby during and/or after treatment the effect of treatment is readily controlled by measurement of DHO in a blood sample and
for repeating treatment according to step c) a decision whether or not further treatment is necessary is taken.

The decision whether or not further treatment is necessary may depend on e.g. the tumour seize found, the number of differentiation arrested blood cell found.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts in a method for treatment of cancer following a dosing schedule comprising
step a) administering the compound to a patient daily for 4 days and
step b) pausing treatment for 2 days and
step c) optionally repeating the schedule defined by steps a) followed by step b) one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 4 days and
step b) pausing treatment for 3 days and
step c) optionally repeating the schedule defined by steps a) followed by step b) one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 5 days and
step b) pausing treatment for 2 days and
step c) optionally repeating the schedule defined by steps a) followed by step b) one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 5 days and
step b) pausing treatment for 3 days and
step c) optionally repeating the schedule defined by steps a) followed by step b) one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts in a method for treatment of cancer following a dosing schedule comprising
step a) administering the compound to a patient daily for 4 days and
step b) pausing treatment for 2 days and
step c) optionally repeating the schedule defined by steps a) followed by step b) one or more times,
whereby during and/or after treatment the effect of treatment is readily controlled by measurement of DHO in a blood sample.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 4 days and
step b) pausing treatment for 3 days and
step c) optionally repeating the schedule defined by steps a) followed by step b) one or more times,
whereby during and/or after treatment the effect of treatment is readily controlled by measurement of DHO in a blood sample.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 5 days and
step b) pausing treatment for 2 days and
step c) optionally repeating the schedule defined by steps a) followed by step b) one or more times,
whereby during and/or after treatment the effect of treatment is readily controlled by measurement of DHO in a blood sample.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 5 days and
step b) pausing treatment for 3 days and
step c) optionally repeating the schedule defined by steps a) followed by step b) one or more times,
whereby during and/or after treatment the effect of treatment is readily controlled by measurement of DHO in a blood sample.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts in a method for treatment of cancer following a dosing schedule comprising
step a) administering the compound to a patient daily for 4 days and
step b) pausing treatment for 2 days and
step c) optionally repeating the schedule defined by steps a) followed by step b) one or more times,
whereby during and/or after treatment the effect of treatment is readily controlled by measurement of DHO in a blood sample and
for repeating treatment according to step c) a decision whether or not further treatment is necessary is taken.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 4 days and
step b) pausing treatment for 3 days and
step c) optionally repeating the schedule defined by steps a) followed by step b) one or more times,
whereby during and/or after treatment the effect of treatment is readily controlled by measurement of DHO in a blood sample and for repeating treatment according to step c) a decision whether or not further treatment is necessary is taken.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 5 days and
step b) pausing treatment for 2 days and
step c) optionally repeating the schedule defined by steps a) followed by step b) one or more times,
whereby during and/or after treatment the effect of treatment is readily controlled by measurement of DHO in a blood sample and
for repeating treatment according to step c) a decision whether or not further treatment is necessary is taken.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvate, salts, solvates of salts in a method for treatment of cancer following a dosing schedule comprising
step a) administering the compound to a patient daily for 5 days and
step b) pausing treatment for 3 days and
step c) optionally repeating the schedule defined by steps a) followed by step b) one or more times,
whereby during and/or after treatment the effect of treatment is readily controlled by measurement of DHO in a blood sample and
for repeating treatment according to step c) a decision whether or not further treatment is necessary is taken.

The number of repeated dosing weeks may vary depending on the severity of the disease and the individual condition of the patient.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 4 to 5 days followed by a recovery period of 2 to 3 days, where the compound is not administered, this schedule being optionally repeated one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 4 to 5 days followed by a recovery period of 2 to 3 days, where the compound is not administered, this schedule being optionally repeated one or more times as often as deemed necessary in order to control the disease, achieve its regression or cure the disease mentioned herein.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 4 days followed by a recovery period of 2 days, where the compound is not administered, this schedule being optionally repeated one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 4 days followed by a recovery period of 2 days, where the compound is not administered, this schedule being optionally repeated one or more times as often as deemed necessary in order to control the disease, achieve its regression or cure the disease mentioned herein.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 4 days followed by a recovery period of 3 days, where the compound is not administered, this schedule being optionally repeated one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 4 days followed by a recovery period of 3 days, where the compound is not administered, this schedule being optionally repeated one or more times as often as deemed necessary in order to control the disease, achieve its regression or cure the disease mentioned herein.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 5 days followed by a recovery period of 2 days, where the compound is not administered, this schedule being optionally repeated one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 5 days followed by a recovery period of 2 days, where the compound is not administered, this schedule being optionally repeated one or more times as often as deemed necessary in order to control the disease, achieve its regression or cure the disease mentioned herein.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 5 days followed by a recovery period of 3 days, where the compound is not administered, this schedule being optionally repeated one or more times.

In some embodiments the present invention relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, its solvates, salts, solvates of salts for the manufacture of a medicament for the treatment of a hyperproliferative disease, especially a cancer disease, whereby the dosing schedule is daily administration of the compound for 5 days followed by a recovery period of 3 days, where the compound is not administered, this schedule being optionally repeated one or more times as often as deemed necessary in order to control the disease, achieve its regression or cure the disease mentioned herein.

One aspect of the invention is the use of DHODH inhibitors, especially those disclosed in WO WO2018/077923, and more specifically N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide for the treatment of cancer applying a 4 days on/3 days off dosing schedule or a 5 days on 2 days off dosing schedule and the respective schedule being optionally repeated one or more times; these compounds for use in the treatment of cancer using a 4 days on/3 days off dosing schedule or a 5 days on/2 days off dosing schedule as well as a method of treatment of cancer diseases comprising administering a specific amount of these compounds while using a 4 days on/3 days off dosing schedule or a 5 days on/2 days off dosing schedule and the respective schedule being optionally repeated one or more times.

One aspect of the invention is the use of DHODH inhibitors, especially those disclosed in WO WO2018/077923, and more specifically N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide for the treatment of cancer applying a 4 to 5 days on/2 to 3 days off dosing schedule and the respective schedule being optionally repeated one or more times as often as deemed necessary in order to control the disease, achieve its regression or cure the disease mentioned herein; these compounds for use in the treatment of cancer using a 4 to 5 days on/2 to 3 days off dosing schedule and the respective schedule being optionally repeated one or more times as often as deemed necessary in order to control the disease, achieve its regression or cure the disease mentioned herein, as well as a method of treatment of cancer diseases comprising administering a specific amount of these compounds while using a 4 to 5 days on/2 to 3 days off dosing schedule and the respective schedule being optionally repeated one or more times as often as deemed necessary in order to control the disease, achieve its regression or cure the disease mentioned herein, whereby the specific amount depends on the patient condition and may vary from patient to patient but still within the amount ranges as disclosed herein.

One aspect of the invention is the use of DHODH inhibitors, especially those disclosed in WO WO2018/077923, and more specifically N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide for the treatment of cancer applying a 4 days on/3 days off dosing schedule or a 5 days on/2 days off dosing schedule and the respective schedule being optionally repeated one or more times as often as deemed necessary in order to control the disease, achieve its regression or cure the disease mentioned herein; these compounds for use in the treatment of cancer using a 4 days on/3 days off dosing schedule or a 5 days on/2 days off dosing schedule and the respective schedule being optionally repeated one or more times as often as deemed necessary in order to control the disease, achieve its regression or cure the disease mentioned herein, as well as a method of treatment of cancer diseases comprising administering a specific amount of these compounds while using a 4 days on 3 days off dosing schedule or a 5 days on 2 days off dosing schedule and the respective schedule being optionally repeated one or more times as often as deemed necessary in order to control the disease, achieve its regression or cure the disease mentioned herein, whereby the specific amount depends on the patient condition and may vary from patient to patient but still within the amount ranges as disclosed herein.

The DHODH inhibitor may be administered in a pharmaceutical composition, in particular comprising N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, or a solvate, a tautomer, an N-oxide, a salt, a solvate of a tautomer, a solvate of an N-oxide, a solvate of a salt, particularly a pharmaceutically acceptable salt, or a mixture of same, and one or more excipients, in particular one or more pharmaceutically acceptable excipient(s) a defined herein. Conventional procedures for preparing such pharmaceutical composition in appropriate dosage forms can be utilized.

In some embodiments the DHODH inhibitor may be administered in a pharmaceutical composition, in particular comprising N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, or solvate, a tautomer, an N-oxide, or a salt, a solvate of a tautomer, a solvate of an N-oxide, a solvate of a salt, thereof, particularly a pharmaceutically acceptable salt, or a mixture of same, and one or more excipients, in particular one or more pharmaceutically acceptable excipient(s) as defined herein for using it in a 4 to 5 days on/2 to 3 days off dosing schedule and the respective schedule being optionally repeated one or more times as often as deemed necessary in order to control the disease, achieve its regression or cure the disease mentioned herein.

In some further embodiments the DHODH inhibitor may be administered in a pharmaceutical composition, in particular comprising N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, or a solvate, a tautomer, a salt, a solvate of a tautomer, a solvate of a salt thereof, particularly a pharmaceutically acceptable salt, or a mixture of same, and one or more excipients, in particular one or more pharmaceutically acceptable excipient(s) a defined herein for using it in a 4 days on/3 days off dosing schedule or a 5 days on/2 days off dosing schedule and the respective schedule being optionally repeated one or more times as often as deemed necessary in order to control the disease, achieve its regression or cure the disease mentioned herein.

In other embodiments the DHODH inhibitor may be administered in a pharmaceutical composition as mentioned herein, in particular comprising N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, or a tautomer, thereof, or a mixture of same, and one or more excipients, in particular one or more pharmaceutically acceptable excipient(s) a defined herein for using it in a 4 days on/3 days off dosing schedule or a 5 days on/2 days off dosing schedule and the respective schedule being optionally repeated one or more times as often as deemed necessary in order to control the disease, achieve its regression or cure the disease mentioned herein.

In some embodiments the DHODH inhibitor may be administered in a pharmaceutical composition as mentioned herein, in particular comprising N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, or a solvate thereof, or a mixture of same, and one or more excipients, in particular one or more pharmaceutically acceptable excipient(s) a defined herein.

This composition can be administered in a suitable manner, such as, for example, via the oral, parenteral, pulmonary, nasal, sublingual, lingual, buccal, rectal, vaginal, dermal, transdermal, conjunctival, otic route in a dosing schedule of 4 to 5 days on/2 to 3 days off, more especially in a dosing schedule of 4 days on/3 days off schedule or in a dosing schedule of 5 days on/2 days off.

The present invention furthermore relates to a method of treatment of a hyperproliferative disease, in particular cancer, comprising administering a pharmaceutical composition comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof together with one or more pharmaceutically acceptable excipient(s) in a 4 to 5 days on/2 to 3 days off schedule, more especially in a dosing schedule of 4 days on/3 days off schedule or in a dosing schedule of 5 days on/2 days off.

The present invention furthermore relates to a method of treatment of a hyperproliferative disease comprising administering a DHODH inhibitor, especially as mentioned herein with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times.

The present invention furthermore relates to a method of treatment of a hyperproliferative disease comprising administering a DHODH inhibitor, especially as mentioned herein in a treatment schedule of 4 to 5 days on/2 to 3 days off.

The present invention furthermore relates to a method of treatment of a hyperproliferative disease administering a DHODH inhibitor in a treatment schedule of 4 to 5 days on/2 to 3 days off.

The present invention furthermore relates to a method of treatment of a hyperproliferative disease with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times comprising administering a DHODH inhibitor where the DHODH inhibitor is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate or a salt, a solvate of a tautomer or a solvate of a salt thereof.

The present invention furthermore relates to a method of treatment of a hyperproliferative disease with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times comprising administering the DHODH inhibitor in a pharmaceutical composition comprising in addition one or more pharmaceutically acceptable excipient(s).

The present invention furthermore relates to a method of treatment of a hyperproliferative disease with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times comprising administering the DHODH inhibitor in a pharmaceutical composition comprising in addition one or more pharmaceutically acceptable excipient(s) wherein the DHODH inhibitor is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide a tautomer, a solvate or a salt, a solvate of a tautomer or a solvate of a salt or a salt of a tautomer.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times whereby the dosing schedule is 4 days or 96 hrs on/3 days or 72 hrs off.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times whereby the dosing schedule is 5 days or 120 hrs on, 2 days or 48 hrs off.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering the DHODH inhibitor to a patient daily for 4 to 5 days and
step b) pausing treatment for 2 to 3 days and
step c) repeating steps a) and b) one or more times.

The present invention furthermore relates to N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a method of treatment of a hyperproliferative disease comprising administering said compound with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering the said compound to a patient daily for 4 to 5 days and
step b) pausing treatment for 2 to 3 days and
step c) repeating steps a) and b) one or more times.

The present invention furthermore relates to N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a method of treatment of a hyperproliferative disease comprising administering said compound with a dosing schedule using daily treatments of equal to 4 days up to 5 days followed by a short off period of equal to 2 days up to 3 days, and the respective schedule being repeated one or more times, comprising the steps of
step a) administering the said compound to a patient daily for 4 to 5 days and
step b) pausing treatment for 2 to 3 days and
step c) repeating steps a) and b) one or more times.

The present invention furthermore relates to N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a method of treatment of a hyperproliferative disease comprising administering said compound with a dosing schedule using daily treatments of a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering the said compound to a patient daily for equal to 96 hrs up to 120 hrs and
step b) pausing treatment for equal to 48 hrs up to 72 hrs and
step c) repeating steps a) and b) one or more times.

The present invention furthermore relates to a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering said compound to a patient daily for 4 to 5 days/or equal to 96 hrs up to 120 hrs and
step b) pausing treatment for 2 to 3 days/or equal to 48 hrs up to 72 hrs and
step c) repeating steps a) and b) one or more times.

The present invention furthermore relates to N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a method of treatment of cancer comprising administering said compound with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, consisting the steps of
step a) administering the said compound to a patient daily for 4 to 5 days/or equal to 96 hrs up to 120 hrs and
step b) pausing treatment for 2 to 3 days/or equal to 48 hrs up to 72 hrs and
step c) repeating steps a) and b) one or more times.

The present invention furthermore relates to N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a method of treatment of cancer comprising administering said compound with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, consisting the steps of
step a) administering the said compound to a patient daily for 4 to 5 days/96 hrs up to 120 hrs and
step b) pausing treatment for 2 to 3 days/48 hrs up to 72 hrs and
step c) repeating steps a) and b) one or more times.

The present invention furthermore relates to a method of treatment of cancer comprising administering a DHODH inhibitor with a dosing schedule using daily treatments of equal to 4 days up to 5 days followed by a short off period of equal to 2 days up to 3 days, to a patient in need thereof and the respective schedule being repeated one or more times, consisting the steps of
step a) administering the DHODH inhibitor to a patient daily for 4 to 5 days and
step b) pausing treatment for 2 to 3 days and
step c) repeating steps a) and b) one or more times.

The present invention furthermore relates to N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a method of treatment of a hyperproliferative disease comprising administering said compound with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering the said compound to a patient daily for 4 to 5 days and
step b) pausing treatment for 2 to 3 days and
step c) repeating steps a) and b) one or more times and,
during and/or after treatment, the effect of treatment is readily controlled by means as disclosed herein.

The present invention furthermore relates to N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a method of treatment of a hyperproliferative disease comprising administering said compound with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering the said compound to a patient daily for 4 to 5 days and
step b) pausing treatment for 2 to 3 days and
step c) repeating steps a) and b) one or more times and,
during and/or after treatment, the effect of treatment is readily controlled by measurement of DHO in a blood sample.

The present invention furthermore relates to a method of treatment of a hyperproliferative disease comprising administering N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering said compound to a patient daily for 4 to 5 days and
step b) pausing treatment for 2 to 3 days and
step c) repeating steps a) and b) one or more times. and,
during and/or after treatment, the effect of treatment is readily controlled by measurement of DHO in a blood sample.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, where the hyperproliferative disease is selected from a hyperplasia affecting the skin, psoriasis, keloids; benign prostate hyperplasia (BPH); a cancer disease; sarcomas; and haematological malignancies.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times whereby the dosing schedule is 4 days or 96 hrs on/3 days or 72 hrs off, and where the hyperproliferative disease is selected from a hyperplasia affecting the skin, psoriasis, keloids; benign prostate hyperplasia (BPH); and a cancer disease of the breast, respiratory tract, brain, reproductive organs, digestive tract, urinary tract, eye, liver, skin, head and neck, thyroid, parathyroid and their distant metastases; sarcomas; haematological malignancies, leukemias, lymphomas, multiple myelomas.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times whereby the dosing schedule is 4 days or 96 hrs on/3 days or 72 hrs off and where the hyperproliferative disease is selected from a hyperplasia affecting the skin, psoriasis, keloids; benign prostate hyperplasia (BPH); and a cancer disease of the breast, respiratory tract, brain, reproductive organs, digestive tract, urinary tract, eye, liver, skin, head and neck, thyroid, parathyroid and their distant metastases; sarcomas; haematological malignancies, leukemias, lymphomas, multiple myelomas.

The present invention furthermore relates to a method of treatment of a hyperproliferative disease comprising administering a DHODH inhibitor with a dosing schedule using daily treatments of equal to 4 days followed by a short off period of equal to 3 days, or a dosing schedule using daily treatments of equal to 96 hrs followed by a short off period of equal to 72 hrs to a patient in need thereof and the respective schedule being repeated one or more times whereby the dosing schedule is 4 days or 96 hrs on/3 days or 72 hrs off and where the hyperproliferative disease is selected from a hyperplasia affecting the skin, psoriasis, keloids; benign prostate hyperplasia (BPH); and a cancer disease of the breast, respiratory tract, brain, reproductive organs, digestive tract, urinary tract, eye, liver, skin, head and neck, thyroid, parathyroid and their distant metastases; sarcomas; haematological malignancies, leukemias, lymphomas, multiple myelomas.

The present invention furthermore relates to a method of treatment of a hyperproliferative disease comprising administering a DHODH inhibitor with a dosing schedule using daily treatments of equal to 5 days followed by a short off period of equal to 2 days, or a dosing schedule using daily treatments of equal to 120 hrs followed by a short off period of equal to 48 hrs to a patient in need thereof and the respective schedule being repeated one or more times whereby the dosing schedule is 5 days or 120 hrs on/2 days or 48 hrs off and where the hyperproliferative disease is selected from a hyperplasia affecting the skin, psoriasis, keloids; benign prostate hyperplasia (BPH); and a cancer disease of the breast, respiratory tract, brain, reproductive organs, digestive tract, urinary tract, eye, liver, skin, head and neck, thyroid, parathyroid and their distant metastases; sarcomas; haematological malignancies, leukemias, lymphomas, multiple myelomas.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times whereby the dosing schedule is 4 days or 96 hrs on/3 days or 72 hrs off, and where the hyperproliferative disease is selected from acute lymphatic leukemia, acute myeloid leukemia, acute promyelocytic leukemia, chronic myelomonocytic leukemia, diffuse large B-cell lymphoma, myeloblastic syndrome, mantle cell lymphoma, T-cell non-Hodgkin lymphoma, Burkitt lymphoma, colorectal cancer, melanoma and ovarian cancer.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times whereby the dosing schedule is 4 days or 96 hrs on/3 days or 72 hrs off, and where the hyperproliferative disease is selected from acute lymphatic leukemia, acute myeloid leukemia, acute promyelocytic leukemia, chronic myelomonocytic leukemia, diffuse large B-cell lymphoma, myeloblastic syndrome, mantle cell lymphoma, T-cell non-Hodgkin lymphoma, Burkitt lymphoma, colorectal cancer, melanoma and ovarian cancer.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times whereby the dosing schedule is 4 days or 96 hrs on/3 days or 72 hrs off, and where the hyperproliferative disease is selected from ALL, AML, APL, CMML, DLBCL, MDS, MCL, T-NHL, Burkitt lymphoma, brain cancer, colorectal cancer, ovarian cancer, pancreas cancer, prostate cancer, glioma, non-small cell lung cancer (NSCLC), small cell lung cancer (SCLC), or melanoma.

The present invention furthermore relates to a method of treatment of a hyperproliferative disease comprising administering a DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times whereby the dosing schedule is 4 days or 96 hrs on/3 days or 72 hrs off where the hyperproliferative disease is selected from ALL, AML, APL, CMML, DLBCL, MDS, MCL, T-NHL, Burkitt lymphoma, brain cancer, colorectal cancer, ovarian cancer, pancreas cancer, prostate cancer, glioma, non-small cell lung cancer (NSCLC), small cell lung cancer (SCLC), or melanoma.

The present invention furthermore relates to a method of treatment as disclosed herein whereby the DHODH inhibitor is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof.

The present invention furthermore relates to a method of treatment as disclosed herein comprising administering a DHODH inhibitor which is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof in a 4 to 5 days on/2 to 3 days off schedule for the treatment of a hyperproliferative disease.

The present invention furthermore relates to a method of treatment as disclosed herein comprising administering a DHODH inhibitor which is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof in a 4 to 5 days on/2 to 3 days off schedule for the treatment of cancer.

The present invention furthermore relates to a method of treatment as disclosed herein comprising administering a DHODH inhibitor which is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof in a 4 to 5 days on/2 to 3 days off schedule for the treatment of a hyperproliferative disease selected from is ALL, AML, APL, CMML, DLBCL, MDS, MCL, T-NHL, Burkitt lymphoma, brain cancer, colorectal cancer, ovarian cancer, pancreas cancer, prostate cancer, glioma, non-small cell lung cancer (NSCLC), small cell lung cancer (SCLC), or melanoma.

The present invention furthermore relates to a method of administration of a pharmaceutical composition comprising N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof together with one or more pharmaceutically acceptable excipient(s) in a 4 to 5 days on/2 to 3 days off schedule, more especially in a 4 days on/3 days off schedule or in a 5 days on/2 days off schedule.

The present invention furthermore relates to a method of administration of a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof together with one or more pharmaceutically acceptable excipient(s) in a 4 to 5 days on/2 to 3 days off schedule, more especially in a 4 days on/3 days off schedule or in a 5 days on/2 days off schedule for treatment of a hyperproliferative disease, especially cancer.

The present invention furthermore relates to a method of administration of a pharmaceutical composition comprising N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof together with one or more pharmaceutically acceptable excipient(s) in a 4 to 5 days on/2 to 3 days off schedule, more especially in a 4 days on/3 days off schedule or in a 5 days on/2 days off schedule for treatment of a hyperproliferative disease, especially cancer.

The present invention furthermore relates to a method of administration of a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 4 to 5 days on/2 to 3 days off.

The present invention furthermore relates to the use of a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for the preparation of a medicament for use in a treatment schedule of 4 to 5 days on/2 to 3 days off.

The present invention furthermore relates to the use of a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for the preparation of a medicament for use in a treatment schedule of 4 to 5 days on/2 to 3 days off for the treatment of a hyperproliferative disease, especially cancer.

The present invention furthermore relates to the use of a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for the preparation of a medicament for use in method of treatment of a hyperproliferative disease with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times.

The present invention furthermore relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for the preparation of a medicament for use in a method of treatment of a hyperproliferative disease comprising administering said compound with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering the said compound to a patient daily for 4 to 5 days and
step b) pausing treatment for 2 to 3 days and
step c) repeating steps a) and b) one or more times.

The present invention furthermore relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for the preparation of a medicament for use in a method of treatment of a hyperproliferative disease comprising administering said compound with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering the said compound to a patient daily for 4 to 5 days and
step b) pausing treatment for 2 to 3 days and
step c) repeating steps a) and b) one or more times and,
during and/or after treatment, the effect of treatment is readily controlled by measurement of DHO in a blood sample and/or by measuring the count ratio of normal cells to differentiation arrested cells in a blood sample and/or by non-invasive methods of imaging a solid tumor enabling measuring the seize of the remaining tumor.

The present invention furthermore relates to the use of N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for the preparation of a medicament for use in a method of treatment of a hyperproliferative disease comprising administering said compound with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, comprising the steps of
step a) administering the said compound to a patient daily for 4 to 5 days and
step b) pausing treatment for 2 to 3 days and
step c) repeating steps a) and b) one or more times and,
during and/or after treatment, the effect of treatment is readily controlled by measurement of DHO in a blood sample.

The present invention furthermore relates to a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 4 to 5 days on/2 to 3 days off.

The present invention furthermore relates to a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 4 to 5 days on/2 to 3 days off for the treatment of a hyperproliferative disease, especially cancer.

The present invention furthermore relates to a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 4 days on/3 days off for the treatment of a hyperproliferative disease, especially cancer, more especially AML or MCL.

The present invention furthermore relates to a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 4 days on/3 days off for the treatment of a hyperproliferative disease, especially cancer, more especially AML.

The present invention furthermore relates to a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 4 days on/3 days off for the treatment of a hyperproliferative disease, especially cancer, more especially MCL.

The present invention furthermore relates to a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 5 days on/2 days off for the treatment of a hyperproliferative disease, especially cancer, more especially MCL or colorectal cancer.

The present invention furthermore relates to a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 5 days on/2 days off for the treatment of a hyperproliferative disease, especially cancer, more especially MCL.

The present invention furthermore relates to a DHODH inhibitor, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate, a salt, a solvate of a tautomer or a solvate of a salt thereof for use in a treatment schedule of 5 days on/2 days off for the treatment of a hyperproliferative disease, especially cancer, more especially colorectal cancer.

In some embodiments the hyperproliferative disease is selected from hyperplasia affecting the skin, psoriasis, keloids; benign prostate hyperplasia (BPH); and a cancer disease of the breast, respiratory tract, brain, reproductive organs, digestive tract, urinary tract, eye, liver, skin, head and neck, thyroid, parathyroid and their distant metastases; sarcomas; haematological malignancies, leukemias, lymphomas, multiple myelomas.

In some embodiments the hyperproliferative disease is cancer, especially the cancer disease is selected from breast cancer, colorectal carcinoma, gastric cancer, glioblastoma, gliosarcoma, glioma, head & neck cancer, hepatocellular carcinoma, lung cancer, leukemia, acute myeloid leukemia, lymphoma, multiple myeloma, neuroblastoma, ovarian cancer, pancreatic cancer, prostate cancer, renal cell carcinoma, and sarcoma or being any single cancer disease as mentioned herein.

In some embodiments the hyperproliferative disease is cancer, especially the cancer disease is selected from ALL, AML, APL, CMML, DLBCL, MDS, MCL, T-NHL, Burkitt lymphoma, breast cancer, brain cancer, colorectal cancer, glioma, lung cancer, melanoma, ovarian cancer, pancreas cancer, and prostate cancer.

In some embodiments the hyperproliferative disease is cancer, especially the cancer disease is selected from cancer is selected from ALL, AML, APL, CMML, DLBCL, MDS, MCL, T-NHL, colorectal cancer, melanoma and ovarian cancer.

In some embodiments the hyperproliferative disease is cancer, especially the cancer disease is ALL, AML, APL, CMML, DLBCL, MDS, MCL, T-NHL, Burkitt lymphoma, brain cancer, colorectal cancer, ovarian cancer, pancreas cancer, prostate cancer, glioma, non-small cell lung cancer (NSCLC), small cell lung cancer (SCLC), or melanoma.

In some embodiments the hyperproliferative disease is cancer, especially the cancer disease is Acute lymphatic leukemia (ALL), acute myeloid leukemia (AML), acute promyelocytic leukemia (APL), chronic myelomonocytic leukemia (CMML), diffuse large B-cell lymphoma (DLBCL), myeloblastic syndrome (MDS), mantle cell lymphoma (MCL), T-cell non-Hodgkin lymphoma (T-NHL), Burkitt lymphoma, colorectal cancer, lung cancer, melanoma, ovarian cancer and prostate cancer.

In some embodiments the hyperproliferative disease is cancer, especially the cancer disease is acute lymphatic leukemia (ALL), acute myeloid leukemia (AML), acute promyelocytic leukemia (APL), chronic myelomonocytic leukemia (CMML), diffuse large B-cell lymphoma (DLBCL), myeloblastic syndrome (MDS), mantle cell lymphoma (MCL), T-cell non-Hodgkin lymphoma (T-NHL), Burkitt lymphoma.

In some embodiments the hyperproliferative disease is cancer, especially the cancer disease is a hematological cancer, a lymphoma or a solid tumor, especially AML, mantle cell lymphoma or colorectal cancer.

In some embodiments the hyperproliferative disease is cancer, and the cancer disease is selected from AML, MCL, and colorectal cancer

In some embodiments the hyperproliferative disease is cancer, especially the cancer disease is leukemia.

In some embodiments the hyperproliferative disease is cancer, especially the cancer disease is acute lymphatic leukemia (ALL).

In some embodiments the hyperproliferative disease is cancer, especially the cancer disease is acute myeloid leukemia (AML).

In some embodiments the hyperproliferative disease is cancer, especially the cancer disease is acute promyelocytic leukemia (APL).

In some embodiments the hyperproliferative disease is cancer, especially the cancer disease is chronic myelomonocytic leukemia (CM M L).

In some embodiments the hyperproliferative disease is cancer, especially the cancer disease is diffuse large B-cell lymphoma (DLBCL).

In some embodiments the hyperproliferative disease is cancer, especially the cancer disease is myeloblastic syndrome (MDS).

In some embodiments the hyperproliferative disease is cancer, especially the cancer disease is mantle cell lymphoma (MCL) T.

In some embodiments the hyperproliferative disease is cancer, especially the cancer disease is lymphoma.

In some embodiments the hyperproliferative disease is cancer, especially the cancer disease is T-cell non-Hodgkin lymphoma (T-NHL).

In some embodiments the hyperproliferative disease is cancer, especially the cancer disease is Burkitt lymphoma.

In some embodiments the hyperproliferative disease is cancer, especially the cancer disease is breast cancer. In some embodiments the hyperproliferative disease is cancer, especially the cancer disease is brain cancer.

In some embodiments the hyperproliferative disease is cancer, especially the cancer disease is colorectal cancer, lung cancer, melanoma, ovarian cancer, and prostate cancer. In some embodiments the hyperproliferative disease is cancer, especially the cancer disease is colorectal cancer. In some embodiments the hyperproliferative disease is cancer, especially the cancer disease is lung cancer, including, but not limited to non-small cell lung cancer and small cell lung cancer. In some embodiments the hyperproliferative disease is cancer, especially the cancer disease is melanoma. In some embodiments the hyperproliferative disease is cancer, especially the cancer disease is ovarian cancer. In some embodiments the hyperproliferative disease is cancer, especially the cancer disease is prostate cancer. The compounds disclosed in WO WO2018/077923, more specifically N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, are useful for treatment of diseases or conditions, including chronic diseases, of humans or other species which can be treated or prevented by inhibition of DHODH by a 4 to 3 days on/2 to 3 days off dosing schedule, especially a 4 days on 3 days off dosing schedule or a 5 days on 2 days off dosing schedule. These diseases or conditions include (1) inflammatory or allergic diseases such as systemic anaphylaxis and hypersensitivity responses, drug allergies, insect sting allergies and food allergies, (2) inflammatory bowel diseases, such as Crohn's disease, ulcerative colitis, ileitis and enteritis, (3) vaginitis, (4) psoriasis and inflammatory dermatoses such as dermatitis, eczema, atopic dermatitis, allergic contact dermatitis and urticaria, (5) vasculitis, (6) spondyloarthropathies, (7) scleroderma, (8) asthma and respiratory allergic diseases such as allergic asthma, allergic rhinitis, allergic conjunctivitis, hypersensitivity lung diseases and the like, and (9) autoimmune diseases, such as arthritis (including rheumatoid and psoriatic), systemic lupus erythematosus, type I diabetes, glomerulonephritis and the like, (10) graft rejection (including allograft rejection and graft-v-host disease), (11) other diseases in which undesired inflammatory responses are to be inhibited, e.g., atherosclerosis, myositis, neurological disorders such as stroke, ischemic reperfusion injury, traumatic brain injury and closed-head injuries, neurodegenerative diseases (e.g., Parkinson's disease), multiple sclerosis, Alzheimer's disease, encephalitis, meningitis, osteoporosis, gout, hepatitis, nephritis, gall bladder disease, sepsis, sarcoidosis, conjunctivitis, otitis, chronic obstructive pulmonary disease, sinusitis and Behcet's syndrome, and (12) immune diseases or conditions.

In another aspect, the present invention provides methods for treating or preventing viral infections in a subject, the methods comprising administering to a subject having or at risk of developing a viral infection a therapeutically effective amount of a compounds disclosed in WO WO2018/077923, more specifically N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide a tautomer, a solvate, a salt, a solvate of a tautomer or a solvate of a salt thereof by using a 4 to 3 days on/2 to 3 days off dosing schedule, especially a 4 days on 3 days off dosing schedule or a 5 days on 2 days off dosing schedule the respective schedule being optionally repeated one or more times.

In yet another aspect, the present invention provides methods of treating or preventing Malaria, the method comprising administering to a subject having or at risk of developing Malaria a therapeutically effective amount of a compounds disclosed in WO WO2018/077923, more specifically N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide a tautomer, a solvate, a salt, a solvate of a tautomer or a solvate of a salt thereof by using a 4 to 3 days on/2 to 3 days off dosing schedule, especially a 4 days on 3 days off dosing schedule or a 5 days on 2 days off dosing schedule the respective schedule being optionally repeated one or more times.

These disorders have been well characterized in humans, but also exist with a similar etiology in other mammals, and can be treated by administering pharmaceutical compositions comprising compounds disclosed in WO WO2018/077923, more specifically N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide a tautomer, a solvate, a salt, a solvate of a tautomer or a solvate of a salt thereof, by using a 4 to 3 days on/2 to 3 days off dosing schedule, especially a 4 days on 3 days off dosing schedule or a 5 days on 2 days off dosing schedule the respective schedule being optionally repeated one or more times.

The total amount of the active ingredient, especially N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide a tautomer, a solvate, a salt, a solvate of a tautomer or a solvate of a salt thereof, to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg (e.g. about 0.5 mg to about 5 mg, about 5 mg to about 50 mg, about 50 mg to about 500 mg, about 500 mg to about 1500 mg, etc.) of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

The compound is administered systemically and hence side effects should be independent of the disease.

Experimental Section

In Vivo Methods

Tumor cells were injected subcutaneously into the flank of the mice. Treatment was initiated at a predefined tumor size after cell inoculation. BAY 2402234 was applied orally (p.o.) at various dose levels and schedules (daily or intermittent). BAY 2402234 was prepared in solution in PEG400/ETOH 9/1. Animal body weight during the study was monitored at least twice weekly. Subcutaneous tumors were measured with a caliper (length and width) at least twice a week, depending on the doubling time of the tumor. Values are presented as volume. Efficacy was calculated on a specific day according to the formula: T/C (treatment/control ratio)=(mean tumor volume from the treated group/mean tumor volume from the vehicle group). Tumor volume was calculated using (width²×length)/2.

### Example 1

AML THP1 cells were inoculated in the flank of SCID mice and 16 days after implantation tumors were measured and animals were randomized at an average tumor volume of around 100 mm³. Treatment with DHODH Inhibitor BAY2402234 was started at time of randomization. Either daily dosing of 5 mg/kg or 10 mg/kg at a 4 days on/3 days off schedule was used and the drug was given orally in 90/10 PEG400/EtOH formulation. Both doses are close to the maximal tolerated dose for the used schedule and mouse strain. The daily dosing sums up to a total weekly dose of 35 mg/kg, while the 4 days on/3 days off schedule sums up to a 40 mg/kg weekly dose. Efficacy for both doses are comparable in this model and the treatments lead to stable disease (FIG. 1).

### Example 2

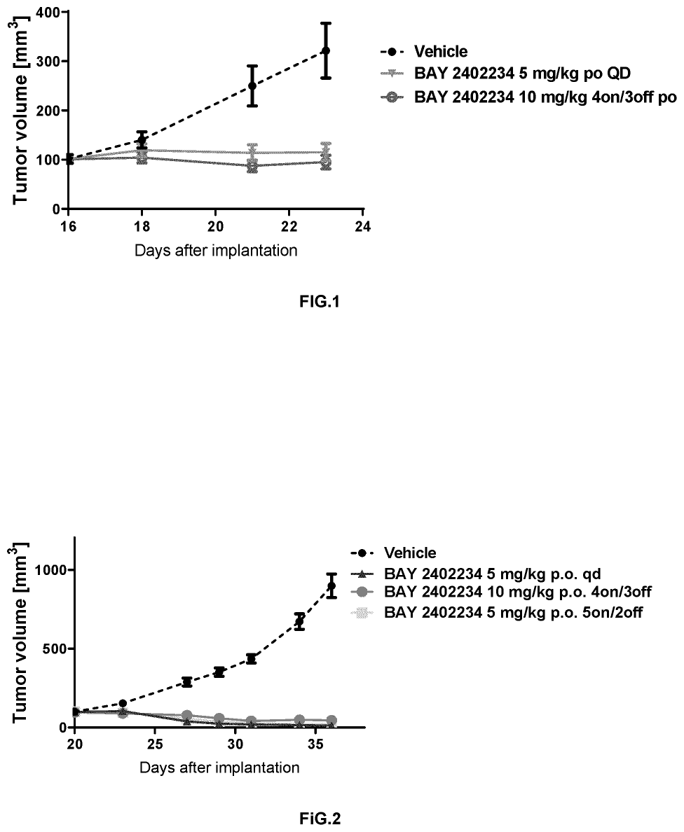

Mantle Cell Lymphoma Z138 cells were inoculated in the flank of SCID mice and 20 days after implantation tumors were measured and animals were randomized at an average tumor volume of around 100 mm³. Treatment with DHODH Inhibitor BAY 2402234 was started at time of randomization. Either daily dosing of 5 mg/kg or 10 mg/kg at a 4 days on/3 days off schedule or 5 mg/kg at a 5 days on 2 days off was used and the drug was given orally in 90/10 PEG400/EtOH formulation. The daily dosing sums up to a total weekly dose of 35 mg/kg, while the 4 days on/3 days off schedule sums up to a 40 mg/kg weekly dose and the 5 days on 2 days off sums up to 25 mg/kg weekly dose. Efficacy for all doses are comparable in this model and the treatments lead to shrinkage of the tumors (FIG. 2)

### Example 3

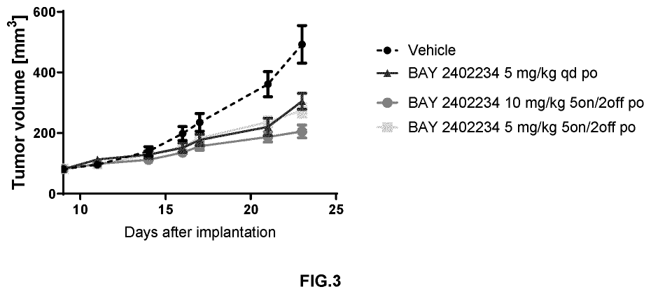

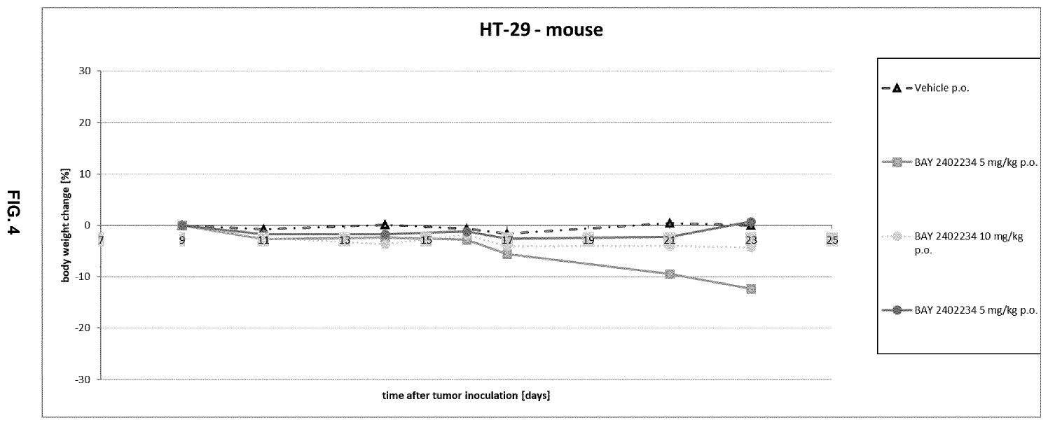

Colorectal cancer cells were inoculated in the flank of mice and 9 days after implantation tumors were measured and animals were randomized at an average tumor volume of around 80 mm³. Treatment with DHODH Inhibitor BAY2402234 was started at time of randomization. Either daily dosing of 5 mg/kg or 10 mg/kg at a 5 days on/2 days off schedule or 5 mg/kg at a 5 days on 2 days off was used and the drug was given orally in 90/10 PEG400/EtOH formulation. The daily dosing sums up to a total weekly dose of 35 mg/kg, while the 5 days on/2 days off schedule sums up to a 50 or a 25 mg/kg weekly dose respectively. Efficacy for the 5 mg/kg schedules are comparable in this model, while the 10 mg/kg intermittent schedule is slightly more efficacious. All the treatments lead to tumor growth delay (FIG. 3). Looking at the body weight loss as a measure of tolerability the 5 mg/kg 5 days on 2 days off was best tolerated followed by 10 mg/kg 5 days on 2 days off and the daily treatment (FIG. 4).

1. A method of treatment of a cancer disease comprising administering the DHODH inhibitor with a dosing schedule using daily treatments of equal to 4 days up to 5 days followed by a short off period of equal to 2 days up to 3 days, or a dosing schedule using daily treatments of equal to 96 hrs up to 120 hrs followed by a short off period of equal to 48 hrs up to 72 hrs to a patient in need thereof and the respective schedule being repeated one or more times, wherein the DHODH inhibitor is N-(2-chloro-6-fluorophenyl)-4-[4-ethyl-3-(hydroxymethyl)-5-oxo-4,5-dihydro-1H-1,2,4-triazol-1-yl]-5-fluoro-2-{[(2S)-1,1,1-trifluoropropan-2-yl]oxy}benzamide, a tautomer, a solvate or a salt, a solvate of a tautomer or a solvate of a salt thereof.

2. The method according to claim 1 for use in a treatment schedule of 4 to 5 days on/2 to 3 days off.

3. The method according to claim 1 comprising administering the DHODH inhibitor in a pharmaceutical composition comprising in addition one or more pharmaceutically acceptable excipient(s).

4. The method according to claim 1 whereby the dosing schedule is 4 days or 96 hrs on/3 days or 72 hrs off.

5. The method according to claim 1 whereby the dosing schedule is 5 days or 120 hrs on, 2 days or 48 hrs off.

6. The method according claim 1, comprising the steps of
step a) administering the DHODH inhibitor to a patient daily for 4 to 5 days and
step b) pausing treatment for 2 to 3 days and
step c) repeating the schedule defined by steps a) followed by step b) one or more times.

7. The method according to claim 6,
whereby during and/or after treatment the effect of treatment is readily controlled by measurement of DHO in a blood sample.

8. The method according to claim 6, whereby during and/or after treatment the effect of treatment is readily controlled by measuring the count ratio of normal cells to differentiation arrested cells in a blood sample.

9. The method according to claim 6, whereby during and/or after treatment the effect of treatment is readily controlled by non-invasive methods of imaging a solid tumor enabling measuring the seize of the remaining tumor.

10. The method according to claim 1, where the cancer disease is selected from a cancer disease of the breast, respiratory tract, brain, reproductive organs, digestive tract, urinary tract, eye, liver, skin, head and neck, thyroid, parathyroid and their distant metastases; sarcomas; haematological malignancies, leukemias, lymphomas, multiple myelomas.

11. The method according to claim 1, where the cancer disease is selected from acute lymphatic leukemia (ALL), acute myeloid leukemia (AML), acute promyelocytic leukemia (APL), chronic myelomonocytic leukemia (CMML), diffuse large B-cell lymphoma (DLBCL), myeloblastic syndrome (MDS), mantle cell lymphoma (MCL), T-cell non-Hodgkin lymphoma (T-NHL), Burkitt lymphoma, brain cancer, colorectal cancer, melanoma, ovarian cancer.

12. The method according to claim 1, where the cancer disease is selected from acute lymphatic leukemia (ALL), acute myeloid leukemia (AML), acute promyelocytic leukemia (APL), chronic myelomonocytic leukemia (CMML), diffuse large B-cell lymphoma (DLBCL), myeloblastic syndrome (MDS), mantle cell lymphoma (MCL), T-cell non-Hodgkin lymphoma (T-NHL), Burkitt lymphoma, brain cancer, colorectal cancer, melanoma, ovarian cancer, pancreas cancer, prostate cancer, non-small cell lung cancer (NSCLC), and small cell lung cancer (SCLC).

13. The method according to claim 1, wherein the administration occurs in a 4 to 5 days on/2 to 3 days off schedule for the treatment of a cancer disease.

14. The method according to claim 1, whereby the cancer disease is selected from AML, MCL, brain cancer and colorectal cancer.

15. The method according to claim 1, whereby the cancer disease is selected from AML, MCL, and colorectal cancer.

16. The method according to claim 13, whereby the cancer disease is selected from AML, MCL, brain cancer and colorectal cancer.

17. The method according to claim 13, whereby the cancer disease is selected from AML, MCL, and colorectal cancer.
